(19) United States
(12) Patent Application Publication
(10) Pub. No.: US 2015/0018282 A1
(43) Pub. Date: Jan. 15, 2015

(54) PREPARATION FOR IMPROVING MEMORY AND LEARNING AND USE THEREOF

(71) Applicant: NAPCO S.AR.L., Luxemburg (LU)

(72) Inventors: Bogdan Lang, Warszawa (PL); Andrzej Lipkowski, Warszawa (PL); Mariusz Sacharczuk, Konstantynow (PL); Elzbieta Salinska, Warszawa (PL)

(73) Assignee: NAPCO S.AR.L., Luxemburg (LU)

(21) Appl. No.: 14/369,246

(22) PCT Filed: Dec. 29, 2012
(86) PCT No.: PCT/EP2012/077100

§ 371 Date: Jun. 27, 2014

(30) Foreign Application Priority Data
Dec. 30, 2011 (PL) P.397623

(51) Int. Cl. C07K 14/47; A23L 1/305

(52) U.S. Cl. CPC C07K 14/47; A23L 1/3053
USPC 514/17.7

(57) ABSTRACT

The invention relates to a preparation for improving memory and learning comprising enzymatic protein hydrolyzate of animal nervous tissue, preferably the tissue of spinal cord of animals for slaughter, while the preferred protein being a myelin protein. The invention also relates to composition for use in treatment of memory disorders, in particular age associated memory disorders and learning impairment. Also disclosed is the method for improving memory and learning.

### FIELD OF INVENTION

The invention relates to a preparation for improving memory and learning and the use thereof.

### BACKGROUND OF INVENTION

Cognitive functions (processes) allow the subject to discover the surrounding reality and determine their behaviour in the environment. Among the cognitive processes memory and learning are often indicated as extremely important. Increasing efficiency of memory and learning processes improves cognitive functions in the subject, which then affects in positive way proper functioning of the subject in the surrounding reality.

Therefore the preparations for improving cognitive activity, in particular those improving memory and learning processes are subject of numerous studies. Known in the prior art are the preparations containing as active ingredients various proteins and peptides, administration of which provides beneficial effect on memory and learning processes.

Patent application WO2011/047204 discloses the beneficial effects of administration of insulin-like growth factor (IGF-II), a nucleic acid encoding a protein of IGF-II, IGF-II peptide, nucleic acid encoding a peptide IGF-II, or mixtures thereof. WO2004/047875 indicates the fibroblast growth factor FGF-18 as a substance which improves memory and learning.

Patent application US2010/0093646 shows a beneficial effect of administration of the dipeptide Leu-Ile to improve memory in a healthy individual, in particular in middle or advanced age, in a steady state clinical condition.

Patent application WO01/80875 discloses a composition for improving memory in humans and animals, comprising as an active ingredient a form of atypical protein kinase C, and in particular protein kinase M zeta, protein kinase C iota/lambda.

WO2010/044529 shows a pharmaceutical composition for improving memory in which the active ingredient used is a protein comprising amino acid sequences in accordance with the GenBank Accession Number XP_{—}193311 “mTMEP and AAH06002” hTMEP.

A positive effect on memory and learning, according to the teaching of the patent application US2010/0081613 also shows a low-dose administration, similar to the physiological level (200 pM), of beta-amyloid peptide.

For the preparation of the prior art formulations it is necessary to use sophisticated methods of synthesis and purification of proteins and peptides. This makes the cost of the active substance relatively high.

### SUMMARY OF THE INVENTION

The aim of the invention is therefore to develop a new formulation that improves memory and learning, which can be used in the manufacture of pharmaceutical products to improve the processes of memory and learning as well as nutritional supplements of such action. It is a further aim of the invention to provide a simple method for preparing such a formulation.

As a result of the study the inventors have surprisingly found a beneficial effect on memory and learning of enzymatic hydrolyte of proteins of the tissue of the central nervous system, and in particular spinal cord obtained from animals for slaughter.

The main component of the spinal cord is the tissue of the nervous system, and the main protein component of the spinal cord are the proteins derived from the lipid-protein substance called myelin, which forms a protective barrier of the nerve tissues.

### DETAILED DESCRIPTION

The present invention relates to enzymatic hydrolysate of proteins of animal tissue of nervous system for use in treatment of memory disorders. Said memory disorders include age associated memory loss or impaired learning.

In one embodiment the enzymatic hydrolysate of proteins of animal tissue of nervous system for use in treatment of memory disorders is an enzymatic hydrolysate of myelin proteins.

In further embodiment the enzymatic hydrolysate of proteins of animal tissue of nervous system for use in treatment of memory disorders is enzymatic hydrolysate of myelin basic protein.

In further embodiment the enzymatic hydrolysate of proteins of animal tissue of nervous system for use in treatment of memory disorders is an enzymatic hydrolysate of a spinal cord tissue of the animal for slaughter, preferably it is an enzymatic hydrolysate of a porcine or lamb spinal cord tissue, more preferably it is an enzymatic hydrolysate of a veal spinal cord tissue.

In further embodiment the enzymatic hydrolysate of proteins of animal tissue of nervous system for use in treatment of memory disorders is obtained in the process of hydrolysis using proteolytic enzyme or enzymes, in particular mammalian digestive enzyme, preferably pancreatine and more preferably pepsin.

In further embodiment the enzymatic hydrolysate of proteins of animal tissue of nervous system for use in treatment of memory disorders is an enzymatic hydrolysate of proteins of animal tissue of nervous system wherein said hydrolysate is obtained in the process of hydrolysis using papain enzyme.

In another embodiment the present invention relates to a composition for use in treatment of memory disorders comprising an effective amount of he enzymatic hydrolysate of proteins of animal tissue of nervous system and one or more carrier or excipient. The use of said composition includes the use in treatment of age associated memory loss and impaired learning.

In further embodiment in the said composition the enzymatic hydrolysate of proteins of animal tissue of nervous system is an enzymatic hydrolysate of myelin basic protein.

In further embodiment in the said composition the enzymatic hydrolysate of proteins of animal tissue of nervous system is an enzymatic hydrolysate of a spinal cord tissue of the animal for slaughter, preferably it is an enzymatic hydrolysate of a porcine or lamb spinal cord tissue, more preferably it is an enzymatic hydrolysate of a veal spinal cord tissue.

In further embodiment in the said composition the enzymatic hydrolysate of proteins of animal tissue of nervous system is obtained in the process of hydrolysis using proteolytic enzyme, in particular mammalian digestive enzyme, preferably pancreatine and more preferably pepsin.

In further embodiment in the said composition the enzymatic hydrolysate of proteins of animal tissue of nervous system is an enzymatic hydrolysate of proteins of animal tissue of nervous system wherein said hydrolysate is obtained in the process of hydrolysis using papain.

In another embodiment the present invention relates to the use of enzymatic hydrolysate of proteins of animal tissue of nervous system as a memory and learning enhancing agent in a food product.

In further embodiment as a memory and learning enhancing agent in a food product the enzymatic hydrolysate of myelin proteins is used.

In another embodiment as a memory and learning enhancing agent in food product the enzymatic hydrolysate of the spinal cord tissue of the animal for slaughter is used, preferably enzymatic hydrolysate of porcine or lamb spinal cord tissue and more preferably enzymatic hydrolysate of veal spinal cord tissue.

In another embodiment, used as a memory and learning enhancing agent in a food product is the hydrolysate of proteins of animal tissue of nervous system obtainable in the process of hydrolysis using a proteolytic enzyme, in particular using papain or a mammalian digestive enzyme, preferably pancreatine, most preferably pepsin.

In another embodiment the hydrolysate of proteins of animal tissue of nervous system, in particular enzymatic hydrolysate of the spinal cord tissue of the animal for slaughter, e.g. porcine, lamb or veal spinal cord, is used as memory and learning enhancing agent in dietary supplement.

In another embodiment the present invention relates to a method of enhancing memory and learning in healthy animal wherein said method involves administrating to the animal an effective amount for enhancing memory and learning of enzymatic hydrolysate of proteins of animal tissue of nervous system.

In further embodiment in the said method said enzymatic hydrolysate is an enzymatic hydrolysate of myelin proteins, preferably myelin basic protein.

In further embodiment in the said method said enzymatic hydrolysate is an enzymatic hydrolysate of the spinal cord tissue of an animal for slaughter, preferably an enzymatic hydrolysate of porcine spinal cord tissue or lamb spinal cord tissue, more preferably an enzymatic hydrolysate of veal spinal cord tissue.

In further embodiment in said method said enzymatic hydrolysate was obtained in the process of hydrolysis using proteolytic enzyme, in particular papain or a mammalian digestive enzyme, preferably pancreatine, most preferably pepsin.

In the context of the present invention, animal tissue of nervous system means the tissue of organs of the central nervous system of vertebrates, excluding humans, in particular brain tissue and spinal cord. These tissues are usually waste products in meat processing plants, and such waste products may be used as a substrate for the hydrolysis process used to obtain enzymatic hydrolysate according to the invention.

The spinal cord obtained from animals for slaughter means in the present context a waste product which is generated in the processing of the carcass in meat processing plants. The spinal cord collected in this process can be used as a substrate of the hydrolysis process described herein immediately after the yield in the processing of the carcass, or it can be frozen for use in hydrolysis after thawing.

The cells of the nervous tissue of the spinal cord, which are responsible for the transmission of stimuli, are protected by a specific substance generally called myelin. The main components of myelin are three myelin proteins: myelin basic protein (MBP), myelin oligodendrocyte protein (MOG) and proteolipid protein (PLP). Methods for isolating myelin proteins are known to those skilled in the art and described in the literature (J. Gagnon, P R Finch, D D Wood, M A Moscarello, Biochemistry, 1971, 10 (25), pp 4756-4763). Myelin proteins in the context of the present invention are to be understood both as a myelin protein in an isolated form as well as the myelin proteins contained in the tissues of the central nervous system, including the spinal cord of animals for slaughter.

Enzymatic hydrolysate improving memory and learning is to be understood in the context of the present invention as a hydrolysate of which the effectiveness in improving memory and learning can be demonstrated by use in animal models well known in the art and used for this purpose, and in particular in a test with a hidden platform (Morris Water Maze MWM, Morris, R. 1984. J. Neurosci. Methods 11, 47-60) and the model of passive avoidance of negative taste stimuli in day-old chicks (Gherkin, A., 1969, Proc Natal. Acad. Sci., USA, 63:1094-1101, Rose, SPR, 1991, Andrew, R J (ed.) Behavioural and Neural Plasticity: The Use of the Domestic Chick as a Model. Oxford University Press, Oxford, 277-304).

The concept of proteolytic enzymes, including the digestive proteolytic enzymes is a concept well known to those skilled in the art and well described in the literature (Alan J. Barrett, Neil D. Rawlings, J F Woessner: Handbook of Proteolytic Enzymes. Oxford: Elsevier Academic Press, 2004). In the context of the present invention mammalian digestive enzymes are to be understood as endogenously occurring enzymes, such as trypsin or pepsin or chemotrypsin or their natural composition—pancreatin.

Method of obtaining an enzymatic hydrolysate according to the invention consists of subjecting fragmented animal nervous tissue, e.g. in the form of spinal cord obtained from animals for slaughter to the process of hydrolysis, wherein the enzyme used for hydrolysis is a proteolytic enzyme. In the method related to the present invention both single proteolytic enzymes, such as for example pepsin or combinations of proteolytic enzymes can be used. Enzymatic hydrolysis of proteins with use of proteolytic enzymes and the method of conducting a process is known in the art and well described (for example, James E Bailey, David F. Ollis Biochemical Engineering Fundamentals, McGraw-Hill Intern. Editions, 1986).

In the context of the present invention, a term “hydrolysate” is to be understood as a product of enzymatic hydrolysis of proteins of animal tissue of the nervous system using proteolytic enzymes.

In a non-limiting, preferred embodiment of the invention, the used proteolytic enzyme is pepsin. In a further preferred embodiment, a solution using 1% wt. pepsin (1:4000) is used, as based on the amount of protein content determined in the spinal cord. Methods for quantitative determination of protein in the sample are known and are commonly used in the art. An example of such a method is the analysis of the nitrogen content in the sample.

For example, in one embodiment the process of hydrolysis with the use of pepsin is carried out at low pH: 1.6-1.9, wherein said pH range can be achieved by the addition of diluted acid, e.g. 1% hydrochloric acid. The preferred temperature of the hydrolysis process is 40° C. In another example the process of hydrolysis may be carried out using papain enzyme in pH of 6.9. In yet another example the process of hydrolysis may be carried out using pancreatine enzyme in pH of 8.2.

According to preferred variant of the process, after 5 hours the enzyme is deactivated by heating the reaction mixture to 80° C., after which the mixture is cooled. The remaining precipitate is filtered and washed with 0.5 part of water. The filtrates are combined and washes are frozen and lyophilized.

Preferably, before hydrolysis, the spinal cord is subjected to a process of separation of low molecular weight fractions. The initial separation of low molecular weight substances allows for a cleaner final product. In one embodiment, said separation is achieved by mixing the ground spinal cord with two volumes of ethanol. Although ethanol may optionally be replaced by other alcohols (hydrated or not), there is a possibility of alcohol residues remaining in the final product. For this reason, it is preferable to use a non-toxic alcohol. In a preferred embodiment, the alcohol solution used is a 60% aqueous solution of ethanol, and the spinal cord remains dispersed in a solution of ethanol for 24 hours at 5° C., after which hydrolysis substrate is separated from the alcohol phase by centrifugation, while the residual alcohol is removed by evaporation from the residue.

The studies on the use of two different animal models have confirmed the positive effect on memory and learning of administration of a preparation containing enzymatic hydrolyzate of animal tissue of nervous system obtained from spinal cord obtained from various animals as well as hydrolysate of isolated myelin basic protein, all herein referred to as “hydrolysate according to invention”.

Studies carried out in mice using a test with a hidden platform (Morris Water Maze MWM) confirmed the positive effect of administration of the hydrolysate according to invention on learning: chronic injections of the hydrolysate for a period of nine days resulted in reducing the time required for animals to find the platform. This positive effect was found in both young mice (2 months) and old mice (16 months). Similarly, a positive effect on learning was found in mice when hydrolysate according to the invention was administered by oral route in the form of 1% by weight of an aqueous solution of the hydrolysate. Oral supplementation of an aqueous solution of hydrolysate according to invention also demonstrated the beneficial effect of administration of the hydrolysate, but its effect was particularly evident in the old mice, which in the final stage of experiment achieved similar results to the young mice.

The studies in mice have confirmed the positive effect of administration of the hydrolysate according to the invention on memory. Comparative study in old mice of the control group showed significant deterioration in memory processes as compared to young mice with similar controls. Intraperitoneal injections of the formulation according to the invention in old mice significantly increased memory capacity of short-term (a day after the removal of the platform) and the long-term (on day seventeen after removal of the platform) to a similar level as in young mice. Oral supplementation with 1% aqueous hydrolysate of the invention has confirmed its effectiveness in the process of memory formation. Similarly as in the intraperitoneal injection, oral administration allowed to keep the memory formation ability in the old mice at a level similar to young mice. This effect was even greater in the second series of the tests (17 days without platform) as compared to the first session on the first day performed after removing the platform. Presumably, the differences observed were mainly due to deterioration of long-term memory of old mice in the control group.

The results obtained in relation to memory processes closely correlated with the results on the learning ability which gives an indication of a very strong pharmacological effect of the hydrolyzate with respect to both learning and memory processes.

Administration of the hydrolysate according to the invention did not present any adverse effects on the health of the animals, and this holds true for both forms of administration: intraperitoneal injection nor oral administration.

The positive impact of the product on memory has also been confirmed in the independent studies based on a different animal model. Used was the model of passive avoidance of negative taste stimuli in day-old chicks. The obtained results clearly show the beneficial effects of administration of the hydrolysate according to invention on training in one-day old chicks.

In one embodiment the hydrolysate can be used as an active ingredient from the group of pharmaceutical agents used in treatment of memory disorders, in particular, but not limited to memory disorders and impaired learning due to aging. Hydrolysate can be used in combination with acceptable additives, in particular pharmaceutically acceptable carriers and excipients which are conventionally used in pharmaceutical practice and are well known in the art. The product may be subjected to known ways of providing for a solid or liquid pharmaceutical compositions, which can be administered both orally or parenterally, or other way where appropriate. The dose of the hydrolysate case can be adjusted by one skilled in the art to achieve the effective amount corresponding to the particular case and desired application.

In general, the enzymatic hydrolysate of the present invention may be administered based upon the dose effective to enhance memory and learning. Such effective amount of the hydrolysate will generally be in a daily dose in a range from about 0.02 to about 30 mg per kilogram of body weight per day.

In another embodiment the hydrolysate can be used as an ingredient in a food product. The demonstrated effect of improving memory and learning in the subjects proves that the hydrolysate of the invention can be used to manufacture food products for enhancing memory and learning, in particular supplements of the nutraceutical type, i.e. a food supplements which bring health and medical benefits. The test results in animal models suggest that the hydrolysate of the invention is particularly suitable for administration in the elderly subjects.

In case of the use of the hydrolysate of the invention for producing food supplement, it is possible to add well-known in the food industry auxiliary materials. They can be selected for their particular properties and thus allow to produce a suitable composition. Such materials are well described in the literature and their selection does not go beyond the routine operation of someone skilled in the art.

Hydrolysate according to present invention demonstrates desired beneficial effect on memory and learning. Additionally, the solution according to invention is characterized by simplicity of the manufacturing process of the hydrolysate, as well as an easy availability and low cost of the substrate material, which is essentially a waste material. The simplicity of the technology of producing the hydrolysate according to invention and the low cost of raw materials ensure economy of the hydrolysate production process. The hydrolysate of the present invention is a product derived from a natural material and produced using natural enzymatic processes wherein the produced peptide fragments correspond to those occurring during the natural digestion.

The following is a description of the accompanying figures:

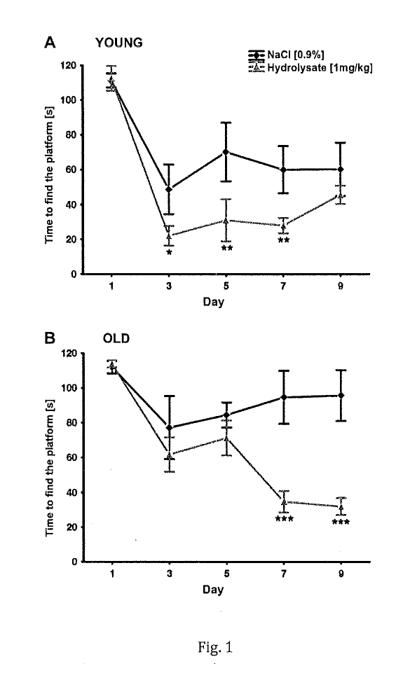

FIG. 1. shows the effect of intraperitoneal injection of the hydrolysate according to invention (named in the diagram as hydrolyzate) at a concentration of [1 mg/kg] body weight on the learning process in young mice (A) and old mice (B).

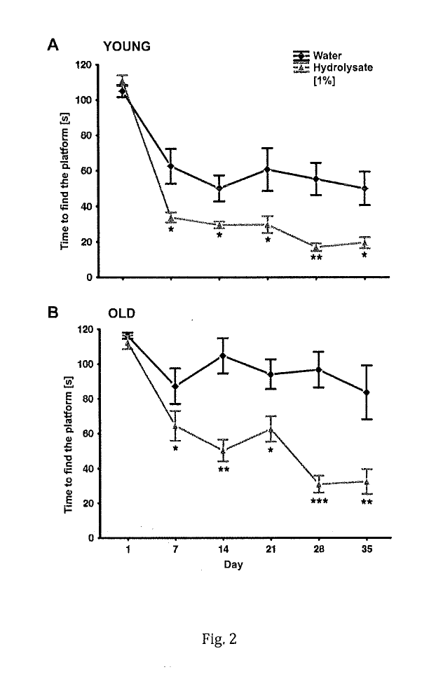

FIG. 2. shows the effect of chronic oral supplementation with aqueous according to invention (named in the diagram as a hydrolyzate) at a concentration of [1%] wt. on learning process of young mice (A) and old mice(B).

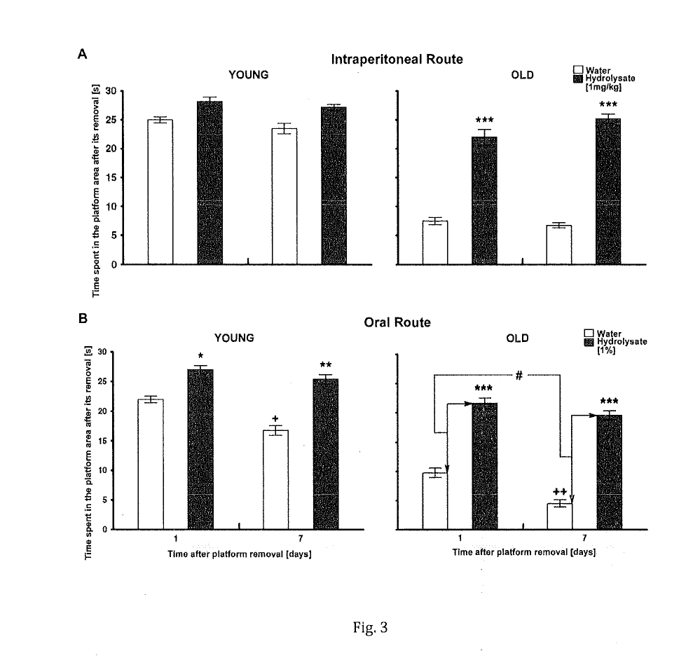

FIG. 3. shows the effect of intraperitoneal injection of the hydrolysate according to the invention (indicated in the diagram as a hydrolyzate) at a concentration of [1 mg/kg] body weight (A) and the oral supplementation (B) on the formation of short-term memory and long-term memory in young mice and old mice.

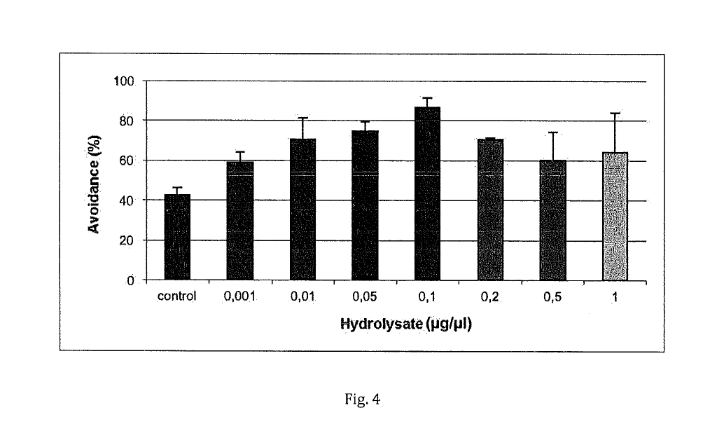

FIG. 4. shows the effect of various concentrations of the hydrolysate according to invention (indicated in the diagram as the hydrolyzate) in the memorization process in the “weak training” model in day-old chicks.

The invention is presented in more detail in the examples set forth below. These examples in no way limit the scope of the invention.

### EXAMPLES

### Example 1

### 1. Process to Obtain Hydrolysate

Fresh porcine spinal cord was milled and mixed with 60% aqueous ethanol solution in the volume ratio 1:2 (substrate: ethanol). After mixing, the material was left for 24 hours at 5° C. The precipitate was then centrifuged, the resulting solid residue was dried from remaining ethanol at room temperature and under reduced pressure (20-50 mm Hg).

The dry solid was fragmented and suspended in 6 parts by weight of water. The suspension was stirred and acidified with 1% hydrochloric acid to pH 1.6, followed by addition of pepsin of activity jFiP 1200/ g in an amount of 0.3% by weight of the starting amount of dry residue. The mixture was heated to 40° C. and the acidity of the reaction mixture was controlled to maintain within pH 1.6-1.9 by the appropriate addition of hydrochloric acid. After 5 hours, the mixture was heated to 80° C. and rapidly cooled to room temperature (21° C.). The remaining precipitate was filtered and washed with 0.5 part of water. The filtrates and washed were combined, frozen and lyophilized.

Residuum contained the product with a content of N=13-13.6% by weight., which indicated peptide content in he product in an amount of about 85 wt %.

The hydrolysate obtained in the above-described manner in the ensuing description showing the results of studies in animal models is referred to as Spineurine.

### Example 2

Study of the effect of Spineurine administration on learning and memory using the swim test with a hidden platform in mice

### 1. The Experimental Material

The experimental material were the outbred male Swiss-Webster mice. The experiments were performed in a experimental lab under standard conditions (temperature 22-23° C. and a 12 hour cycle of light—7:00-19.00 light phase). Provided was unlimited access to water and food (standard food for rodents in the form of pellets from LABOFEED H, Poland: 22% protein (with 1.5% lysine), 5% crude fiber, 4% crude fat, 6.5% crude ash and calories 13.4 kcal/g). Mice were selected randomly to the experimental groups and housed individually in cages (Plexiglas). Each animal was weighed daily and checked for health conditions by a veterinarian. Testing all of the mice was preformed at the same time (between the hours of 9.00 and 14.00). During the experiments the employees who tested the mice were not informed whether the particular mouse had previously been treated with Spineurine. After the end of the experiment the animals were euthanized in accordance with the procedures established for testing of pharmaceuticals by REMONDIS (Ltd., Branch Warsaw, ul. Zawodzie 16, 02-981 Warsaw).

In accordance with the objective of the study, studied mice belonged to two age groups, the first of which were adult mice (7-8 weeks old) with normal cognitive function. The mice in the second age group, were 16-month old mice, which is the period of life in which learning processes are deteriorating, which is a normal process associated with aging. Spineurine effectiveness on learning was assessed for both intraperitoneal injection and oral supplementation in both said age groups. Each of the two age groups consisted of 12 subjects.

Summary of experimental setup is shown in Table 1 below.

TABLE 1
Experimental setup for two age groups of mice

| | | | | Dose per | Volume per | |
|---|---|---|---|---|---|---|
| | No. of | | | kg of body | 10 g. of body | Spineurine |
| Group | subjects | Agent | Route | weight | weight | concentration |
| Mice age group | 12 | Spineurine | Intraperitoneal | 1 mg | 0.1 ml | 0.1 mg/1 ml |
| 7-8 weeks old | | | Injection | | | 0.9% NaCl |
| Mice age group | 12 | NaCl | Intraperitoneal | 0 mg | 0.1 ml | 0 mg/1 ml 0.9% |
| 7-8 weeks old | | | Injection | | | NaCl |
| Control group | | | | | | |
| Mice age group | 12 | Spineurine | Intraperitoneal | 1 mg | 0.1 ml | 0.1 mg/1 ml |
| 16 months old | | | Injection | | | 0.9% NaCl |
| Mice age group | 12 | NaCl | Intraperitoneal | 0 mg | 0.1 ml | 0 mg/1 ml 0.9% |
| 16 months old | | | Injection | | | NaCl |
| Control group | | | | | | |

| | | | | | |
|---|---|---|---|---|---|
| Mice age group | 12 | Spineurine | Oral | Ad libitum | 1% aqueous |
| 7-8 weeks old | | | | | solution |
| Mice age group | 12 | Woda | Oral | Ad libitum | 0% |
| 7-8 weeks old | | | | | |
| Control group | | | | | |
| Mice age group | 12 | Spineurine | Oral | | 1% aqueous |
| 16 months old | | | | | solution |
| Mice age group | 12 | Woda | Oral | | 0% |
| 16 months old | | | | | |
| Control group | | | | | |
| Total | 96 | — | — | | — |

### 2. Spineurine Administration

Intraperitoneal injections of Spieneurine were performed in a dose of 1 mg/kg body weight (bw). Lyophilized Spineurine as obtained in example 1 was dissolved in 0.9% NaCl (0.1 mg/1 ml 0.9% NaCl) and administered in a volume of 0.1 ml/10 g of body weight as intraperitoneal injection. The control groups received injections of 0.9% NaCl in a similar volume.

Oral Spineurine was fed (ad libitum) after its dissolution in water at a concentration of 1%. The control group received pure water (ad libitum). In order to verify a possible taste aversion to Spineurine solution mice were provided simultaneous access to both water and Spienurine solution for a period of three days. During this time, at 12 hours intervals the location of bottles with water and with Spineurine solution was changed and the volume of drunk fluid was measured. Since there was no taste aversion to Spineurine (mice have consumed similar amounts of both—Spineurine solution and water), the Spineurine administration began as the only available fluid (ad libitum).

### 3. The MWM Test Conditions

In order to evaluate the effectiveness of Spineurine as a preparation affecting learning and memory processes, used was a Morris Water Maze MWM test (test with a hidden platform), an animal model which is most commonly used for such purpose. In said test the animal is forced to swim to find the platform which is invisible for the animal, while the animal is free to choose the search strategy (Vorhees and Williams, 2006). Such experiment can be analysed in a simple way just by measuring the execution time of the test, or using more advanced methods, such as based on investigating the spatial navigation strategy (Graziano et al 2003, Patil et al 2008; Steward et al 2011). This method, originally developed for rats (Morris 1981.1984) has been adapted to other species of rodents, especially mice (Upchurch and Wehner 1988). Despite numerous modifications developed by different research teams, the principle of the test is to make the mouse swimming in a tank with filled with water (half a tank), of a diameter of about 100-120 cm and a height of 40-50 cm. In the tank there is a round platform (7-10 cm ø) hidden just below the surface of the water (about 1-2 cm). Also provided are some elements which serve for the mice as orientation marks (Graziano et al 2003). Temperature range used in the MWM test is 20°-36° C.

In the present research the following conditions and parameters were employed: tank diameter—120 cm, platform diameter—10 cm, surface water over the platform (made of gray Plexiglas, with the same colour as the tank)—1.5 cm, water temperature—20° C. Platform during the whole testing period was hidden in one place in the middle of the central part and the edge of the reservoir, an arrangement which is a widely used.

### 4. General Conditions of Testing

### 4.1. Effect of Spineurine Administration on Learning Process

The spatial learning process in the experiment wherein Spineurine was administered via intraperitoneal injections included 9 days of tests performed every other day (a total of 4 tests). The task was to find a platform as soon as possible, following indications from the environment, which were the two bright lights placed at the edge of the tank (parallel to the platform), and the metal wall of 1 m×1 m in size placed by the edge of the tank on the side of the platform. In all experiments, the maximum time allocated for finding the platform was 120 seconds and if within this time the mouse did not found the platform, the animal was removed from the water and kept on the platform for 30 seconds. At the same time in each experiment (day) three attempts were allowed for the mouse to find the platform, performed in 30-minute intervals. In each experiment, the mouse was let to enter the tank from another side (with the head facing the wall of the tank). During each test, immobility time was also measured as an indicator of motor activity.

The effect of oral supplementation with Spineurine on the learning process included six tests (day 0, 7, 14, 21, 28, 35) performed on a weekly basis (35 days). Different time profile for oral administration of Spineurine, i.e significantly longer experiments and intervals between successive tests with different apparent were caused by different Spineurine pharmacokinetics related to two different routes of administration, and in particular, considerable delay in pharmacological effect in oral administration.

### 4.2. Effect of Spineurine Administration on Memory

On the 10^{th} day of intraperitoneal administration of Spineurine, i.e. at the end of learning to find the platform process, mice were subjected to a test for spatial memory, in which the platform was removed. After intraperitoneal injection of Spineurine the mice were let to swim for 120 seconds. During this time the time of motor activity was measured and as well as number of times when the mouse swam over the place where the platform was located during the learning processes was recorded. The experiment included three trials for each mouse, the trails were performed in 30-minute intervals (Kipnis et al 2004, Patil et al 2007). This experiment, showing the effect of Spineurine administration on on long-term memory was repeated on the 17^{th} day after removal of the platform. In the period between 10^{th} and and 17^{th} day mice received injections of Spineurine or 0.9% NaCl, but had not been tested.

In the chronic oral Spineurine supplementation experiment also examined was the long-term effects on memory and in the same manner as in the experiment with intraperitoneal administration of Spineurine, the experiment was carried out in two replications done after a week intervals after cessation of testing, i.e. on 36^{th} and on 43^{rd} day after the start of experiment defining the learning process.

Such procedures for determining long-term memory (without testing between two trials), while they are very important for pharmacological testing of pharmaceutical agents of potential therapeutic interest, they have so far been described only in a few experiments (eg, Tan et al 2006, Patil et al, 2008).

### 4.3. Effect of Spineurine on Anxiety—Raised Plus Maze (Elevated Plus Maze, EPM)

Animal behaviour assessment using the EPM test is commonly used to examine anxiolytic and anxiogenic compounds (Lister, 1990). EPM for mice is constructed in the form of a cross made of black plexiglass having arms of length of 30 cm and a width of 4 cm and elevated 40 cm above the floor (Gorman and Dunn, 1993). Two opposing arms are enclosed by walls 15 cm high (closed arms). The other two arms are open (open arms). Open space in the middle of the cross is defined as the center. Mouse is placed in the center of the EPM head towards the closed arm. Behavior of the individual during the test lasting 6 minutes is analyzed in relation to the time spent in the open arms, closed arm and the center. Also the number of entries into each zone is analysed. EPM test was carried out in a red light conditions after previous three-hour habituation of animals to the lighting conditions.

### 4.4. Effect of Spineurine Administration on the Level of Post-Stress Analgesia

The level of post-stress analgesia was determined by subjecting mice to 3 minutes of swim stress (swim stress-induced analgesia—SSIA) in water at 20° C. followed by examination of the change in pain sensitivity in the hot plate test (Hot Plate—HP, Panock et al 1986a, b). Once removed from water, the tested mouse was let to dry for two minutes in a box filled with lignin paper after which the mouse was placed on a metal plate heated with water of 56° C. and controlled by a thermostat. The pain threshold was expressed as the time (measured in seconds) which lapsed from the moment of placing the mouse on the plate until the response to the thermal stimulus in the form of characteristic raise of the rear limb. In this way, the skin temperature at which the pain receptors are stimulated can be established. It is a commonly used measurement technique that reduces to the minimum the discomfort of animals (Dubner 1989, Thompson 1990).

The extent of post-stress analgesia of animals in the control group and the Spineurine group expressed as % MPE (maximum possible effect) was calculated according to the following formula:

$$\frac{{P}_{k} - {P}_{p}}{( cut off - {P}_{p} ) \times 100}$$

wherein:
Pp—pain latency before swimming
Pk—pain latency after swimming
cut-off (threshold time): 60 s

### 4.5 Statistical Analysis

For the calculations ANOVA 3-factorial analysis of variance with repetition was used, assuming as main effects: Spineurine, time and age of the animals. Bonferroni post-hoc test was used for subsequent analysis for comparison at a given point in time.

### 5. Results

### 5.1 Effect of Spineurine Administration on the Learning Process

The results showing the effect of intraperitoneal injection of Spineurine [1 mg/kg] on the learning process of young mice (A) and old mice (B) are shown in FIG. 1 (intraperitoneal) and FIG. 2 (oral administration). Summary results are presented in Table 2

TABLE 2

| | Number | | | Effect on Learning | Effect on learning |
|---|---|---|---|---|---|
| | Of | | | (relates to control | (relates to study |
| Group | subjects | Agent | Route | group) | group) |
| Mice age group | 12 | Spineurine | Intraperitoneal | Significant | Significant |
| 7-8 weeks old | | | Injection | | |
| Study group | | | | | |
| Mice age group | 12 | NaCl | Intraperitoneal | Significant | N/A |
| 7-8 weeks old | | | Injection | | |
| Control group | | | | | |
| Mice age group | 12 | Spineurine | Intraperitoneal | Insignificant | Significant |
| 16 months, old | | | Injection | | |
| Study group | | | | | |
| Mice age group | 12 | NaCl | Intraperitoneal | Insignificant | N/A |
| 16 months old | | | Injection | | |
| Control group | | | | | |
| Mice age group | 12 | Spineurine | Oral | Significant | Significant |
| 7-8 weeks old | | | | | |
| Study group | | | | | |
| Mice age group | 12 | Water | Oral | Significant | N/A |
| 7-8 weeks old | | | | | |
| Control group | | | | | |
| Mice age group | 12 | Spineurine | Oral | Significant | Significant |
| 16 months old | | | | | |
| Study group | | | | | |
| Mice age group | 12 | Water | Oral | Significant | N/A |
| 16 months old | | | | | |
| Control group | | | | | |
| Total | 96 | — | — | — | — |

N/A—not applicable

The results of research show that chronic injections of Spineurine for nine days decreased the time required to find the platform for both young mice (2 months old) and old (16 months old). During the process of learning to find the platform phasic behaviour was observed expressed in random swimming and looking for opportunities to get out of the water in the first period of experiment, which turned in the later stage into deliberate search for the platform, even after its removal. Such process is commonly observed and described by other authors (e.g., Balschun et al 2003).

In young mice the learning process was affected by both endogenous factors (improved memory in control mice receiving 0.9% NaCl) and by the effect of administration of Spineurine (Spineurine experimental groups of mice quicker learned to locate the platform).

In old mice the improvement in the learning process was observed only in the groups receiving Spineurine. Mice in this group after seven days of injection were able to find the platform in the time similar to that of young mice. This indicates a high efficacy of the hydrolysate according to invention in the potential treatment of memory disorders resulting from aging process. Old mice, treated with water during the whole period of experimental phase showed predominant random swimming and swimming around the pool with long periods of immobility (helplessness), i.e. the behaviour characteristic of the aforementioned initial phase (Janus 1984, Brody and Holtzman 2006).

Oral supplementation with Spineurine also showed its significant, positive impact on the learning process. When comparing the groups receiving Spineurine and control (receiving plain water), Spineurine effectiveness was particularly evident in old mice. These differences are due to the negligible effect of endogenous learning in the old mice (i.e. effect independent of Spineurine administration. At the same time improvement in the ability to find the platform was evident in mice receiving Spineurine, particularly in the last two weeks of the experiment. In the final part of experiment, mice receiving Spineurine achieved similar results as the young mice (about 20 seconds to find the platform).

Just as in the experiment with the use of intraperitoneal injection of the hydrolysate according to invention, during the process of learning to find the platform phasic behaviour was observed expressed in random swimming and looking for opportunities to get out of the water in the first period of experiment, which turned in the later stage into deliberate search for the platform, even after its removal.

Old mice, treated with water during the whole period of experimental phase showed predominant random swimming and swimming around the pool with long periods of immobility (helplessness).

### 5.2 Effect of Spineurine Administration on Memory

The results showing the effect of intraperitoneal injection of Spineurine [1 mg/kg] (A) and its oral supplementation (B) on the process of creating a short-term memory and long-term memory in young and old mice are presented in FIG. 3 wherein:

Notation *, **, *** indicate a significant effect of Spineurine on improvement of memory processes (study groups receiving Spineurine vs. Control groups receiving water) with a probability of, respectively, 0.05, 0.01, 0.001.

+, ++ indicate shortening of the time of presence in the area from where platform was removed in the 7^{th} day following its removal, as compared to day 1. It shows that in the absence of recalling tests long-term memory deteriorates.

# indicates significantly greater effect of oral Spienurine supplementation in old mice, in the 7th day of the experiment after removing the platform, compared with day 1. It is the result of interaction: SPINEURINE×time, showing a slowdown in the process of forgetting caused by administration of Spineurine.

Collectively the results are summarized in Table 3

TABLE 3
Summary of the results related to memory processes

| | Number | | | Effect of Spineurine administration on |
|---|---|---|---|---|
| Group | Of subjects | Agent | Route | memory improvement (long-term) |
| Mice age group | 12 | Spineurine | Intraperitoneal | At the limit of statistical significance |
| 7-8 weeks old | | | Injection | (P = 0.059) |
| Study group | | | | |
| Mice age group | 12 | NaCl | Intraperitoneal | N/A |
| 7-8 weeks old | | | Injection | |
| Control group | | | | |
| Mice age group | 12 | Spineurine | Intraperitoneal | Significant |
| 16 months old | | | Injection | |
| Study group | | | | |
| Mice age group | 12 | NaCl | Intraperitoneal | N/A |
| 16 months old | | | Injection | |
| Control group | | | | |
| Mice age group | 12 | Spineurine | Oral | Significant |
| 7-8 weeks old | | | | |
| Study group | | | | |
| Mice age group | 12 | Water | Oral | N/A |
| 7-8 weeks old | | | | |
| Control group | | | | |
| Mice age group | 12 | Spineurine | Oral | Significant |
| 16 months old | | | | |
| Study group | | | | |
| Mice age group | 12 | Water | Oral | N/A |
| 16 months old | | | | |
| Control group | | | | |
| Total | 96 | — | — | — |

n.d.—nie dotyczy

On the first day after platform removal young mice receiving Spineurine via injection showed only a small, non-significant improvement in the recall processes (memory) compared to control group of mice. A similar trend was maintained after a week break in testing and the lack of availability of the platform. This indicates the high efficiency of endogenous memory processes in young mice. Comparative analysis of the control group of young and old mice showed significant deterioration in memory processes in old mice compared to young mice. Intraperitoneal injections of Spineurine in old mice significantly increased memory capacity of short-term (1^{st} day after removal of the platform) and the long-term (7^{th} day after the removal of platform) up to the similar level as that observed in young mice. The results of the memory-related processes closely correlated with learning ability results and thus confirm a very strong pharmacological effect of Spineurine on reducing the negative effects of aging on learning and memory.

Oral supplementation with 1% aqueous Spineurine also confirmed its effectiveness in the processes of memory formation and recall. Similarly as in the intraperitoneal injection route, oral administration of Spineurine in old mice allowed to maintain the ability to create a memory at the level similar to young mice. This effect was even greater in the second series of tests (7^{th} day without platform) as compared to the first session on the first day after platform removal, which was a effect of deterioration of a long-term memory in old mice and strong counteractive effect of Spineurine administration.

### 5.3 Effect Spineurine Administration on Motor Activity and Anxiety Behavior and General Comments

Mice receiving aqueous solution of Spineurine exhibited 6-fold reduction in immobility time in the Morris-Maze Test. Increased physical activity was confirmed in the forced swimming test without platform (FST—forced swim test). While using the elevated plus maze test (EPM), developed to assess anxiety behavior, we excluded the possibility of reducing the immobility time (increased physical activity) as a result of anxiolytic effect of Spineurine, based on which direct effect of Spineurine on motivational processes can be concluded. In addition, using the hot plate test (HP—Hot Pate) Spineurine proved to show no effect on the level of stress caused by swimming and conduction of painful stimuli.

During the experiment with oral supplementation, there was no difference in preferences between Spineurine and water consumption, both in young and old mice. In the ad libitum conditions the daily intake of water and 1% Spineurine was in the range of 100-120 g/kg/day each. After removal of the water container in the groups of mice receiving Spineurine the daily intake was in the range of 180-220 g/kg/day. The Spineurine solution used at the concentration of 1% wt which means administration of 1.8-2.2 g/kg/day of the lyophilised hydrolysate.

Spineurine also had no effect on food intake, which was in the range of 2300-2700 kcal/kg/day, and the only differences are variables that determine the age of the animals and testing days.

Despite the lack of differences in feed intake, old mice with oral supplementation of Spineurine displayed higher body weight in the final phase of the experiment. In case of young mice receiving oral form of Spineurine there was no significant gain in weight. No offect of Spineurine administration using intraperitoneal injection was observed which was due to the relatively short period of the experiment.

### Example 3

### Assessment of Spineurine Administration Using a Model of Passive Avoidance of Negative Taste Stimuli in Day-Old Chicks (Passive Avoidance Task in One-Day Old Chicks)

### 1. The Testing Method

The method is based on avoidance of pecking artificial grain coated with a bitter tasting substance, methyl anthranilate (MeA) (Gherkin, 1969, Rose, 1991). At the beginning of the experiment chicks are presented three times with the white grain of a diameter of 2 mm for 10 seconds every 5 minutes to verify if the animals are eager to peck. Then the animals are presented with the MeA coated metal grain. Only animals showing the correct response to the bitter taste (shaking their head and the whole body, closing eyes and cleaning their beak against the floor) are found eligible for the experiment. Animals are also tested by the presentation of the metal grains not covered by any substance. This test is performed at various times after the first training (from 1 to 24 hours.). Following the presentation of the metal grains chicks are again presented with the white grain. Chicks avoiding pecking the metal grain while pecking white grains during the final test are classified as those that remembered the training.

This method has two alternatives, depending on whether the compounds tested may affect the amplification of the remembering process or rather they are believed to interfere with the memorising process.

Option I—so called weak training—when the metal grain is covered with 10% solution of MeA. Chicks avoid pecking the grain for a few hours. If the action of the test compound enhances memory, chick will avoid pecking the grain, even after 48 hours.
Option II—so-called—strong training—when the grain is covered with 100% MeA solution. Chicks avoid pecking the grain up to 48 hours. If the action of the tested compound interferes with the process of remembering, chicks will peck at the grain metal in just a few hours after administration of the compound.

Tested compounds can be administered by direct intracerebral injection into brain region known for participation in the learning and memory processes (IMM formerly known as IMHV). Skull of day-old chicks is still not ossified, making it easier to perform an injection.

For the injection a Hamilton syringe (10 ml) is used wherein said syringe has a stop mounted on the needle which allows an injection of a predetermined depth. Injections are free hand performed after local anesthesia using lignocainum spray. This procedure is fast and does not cause stress in chicks. The substances are injected into the two hemispheres (not more than 2.5 ul per hemisphere). In basic experiments, the animals are tested usually 24 hours after training. Compounds which are known to penetrate the blood-brain barrier, can also be administered intraperitoneally.

The effect of the test compound is evaluated based on the way the chicks behave at the sight of the metal grain. The effect is also assessed based on the percentage of animals avoiding the pecking amongst the total number of animals in the study group. Said percentage is then compared with the control group.

At the end of the experiment randomly tested was the injection accuracy, performed using macroscopic evaluation.

To confirm whether the observed effect is not due to the injection itself, in the control group the chicks are injected with physiological saline.

Furthermore, an additional test is used, the so-called discrimination test. In said test chicks are presented with the white grain used in the initial training and after a few minutes they are presented with identical grain that previously presented, but now covered with MeA. Chicks should be able to recognize the difference between the grains. The degree of discrimination is assessed by comparing the number of chicks that could distinguish the grain as compared to those not being able to do so.

### 2. Test Conditions

### 2.1 Training

A day-old chicks were placed in aluminium boxes dimensioned 20×25×20 cm, lit and heated to a temperature of 25-28° C. Chicks had constant access to water and feed. At the beginning of the experiment, in order to see if the animals are eager to peck, the chicks were presented with white grain, 2 mm in diameter, three times for 10 seconds each time, every 5 minutes. Then, the animals were presented with a grain of metal covered with diluted (10%), bitter tasting substance—methyl anthranilate (MeA). Only animals showing the correct response to the bitter taste (shaking their head and the whole body, closing eyes and cleaning beak against the floor) were found eligible for further stages of the experiment.

Animals were tested after 24 hours with the presentation of the metal grain not covered by any substance. After the presentation of the metal grain, chicks were again presented with white grain in order to check whether the chick can distinguish the bad tasting grains from others (so called discrimination test). Chicks avoiding pecking the metal grain and pecking only the white grain during the final test were classified as those remembering the training. In this variant of the experiment (weak training) chicks forget about the bad taste of metal grain after 6-8 hours. If the action of the test compound enhances memory, chicks avoid grains even after 48 hours.

To test whether a compound has no negative effect on memory, a control experiment was performed in which the metal grain was soaked in 100% of the MEA (the so-called strong training). Chicks avoid pecking the grain for up to 48 hours. If the action of the test compound interferes with the process of remembering, chicks peck at metal grain in just a few hours after administration of the compound.

### 2.2 Application of Spineurine

Spineurine was administered by direct intracerebral injection into the area of the brain of the chick known to participate in the process of learning and memory (IMM formerly known as IMHV). Spineurine was dissolved in saline (0.9% NaCl) in a concentration selected from the literature.

In experiments the following concentrations were tested: 0.001 μg/μl, 0.01 μg/μl, 0.05 μg/μl, 0.1 μg/μl, 0.2 μg/μl, 0.5 μg/μl, 1 μg/μl. In the said strong training experiment the used concentration was that at which the highest memory enhancement effect was observed, that is 0.1 μg/μl.

For the injection Hamilton syringe (10 ml) was used, wherein said syringe had a needle stop attached which allowed for the injection at specified depth. Injections were performed using a “free hand” method after local anesthesia using lignocainum spray. Chicks injected with 2.5 μl of the solution of the test substance for each hemisphere. The control group consisted chickens that received saline instead of study treatment.

### 2.3. Results

Effect of different concentrations of Spineurine on the process of remembering in the “weak training” model in a day-old chicks is shown in FIG. 4. All the results obtained differ significantly from the controls (p<0.01). Results for each concentration are the average of the three test groups of 15 chicks in each group (n=3×15).

The results indicate that the administration of Spineurine in the concentrations tested significantly increases the memory of training in one-day old chicks, which was demonstrated by an increase in percentage of chicks avoiding pecking grain from 40% in the control group (injection of normal saline) to 80% at a concentration of at Spineurine 0.1 μg/μl. Furthermore, the results show that Spineurine at the most effective concentration determined in “weak training” model, displayed no negative effect on remembering the training wherein 100% of the MEA was used, the described above “strong training” model (the results obtained in this differed from control and the difference was statistically significant (p<0.01)).

Based on the obtained results it can be concluded that Spineurine administration gives positive effect on the remembering process in chicks undergoing training in a weak variant of the experiment (“weak training”). The memory of the bitter taste of metal grains was considerably extended from 6-8 hours to 24 hours, i.e. until the time of testing. This may indicate strengthening of a stimulus well enough to initiate the formation of the so-called long-term memory. In addition, the tested compound—Spineurine—does not interfere with the process of memorizing strong stimuli that trigger long-term memory formation processes. These results may indicate the direct effect of Spineurine on memory formation and its enhancing.

### Example 4

Comparative studies: different protein sources and enzymes used for hydrolysis The present example shows the results obtained using a model of passive avoidance of negative taste stimuli in day-old chicks and related to the effect on memory caused by administration of hydrolysates based on various protein sources and obtained using different enzymes.

### 1. Materials and Methods

### 1.1. Myelin Basis Protein Hydrolysate (Later Referred to as MBP)

Myelin Basic Protein of bovine origin (Sigma-Aldrich) was hydrolysed according to method described in Example 1. Lyophilised hydrolysate was used for tests following the test protocol described in example 3.

### 1.2. Lamb Spinal Cord Protein Hydrolysate (Later Referred to as LSCP)

Fresh spinal cord from lamb was processed and hydrolysed following the method as described in Example 1. Lyophilised hydrolysate was used for tests following to protocol described in example 3.

### 1.3. Veal Spinal Cord Protein Hydrolysate (Later Referred to as VSCP)

Fresh spinal cord from lamb was processed and hydrolysed following the method as described in Example 1. Lyophilised hydrolysate was used for tests following to protocol described in example 3.

### 1.4. Porcine Spinal Cord Protein Papain Hydrolysate

Fresh porcine spinal cord was processed and hydrolysed following the method described in Example 1, except that papain (Sigma-Aldrich) was used for hydrolysis instead of pepsin while the pH of the reaction was maintained at 6.9. After hydrolysis which was carried out using the time and temperature parameters the same as in Example 1, the reaction solution was acidified to pH 2.5 by use of diluted hydrochloric acid and heated to 80° C. to deactivate the enzyme. The product of hydrolysis was further processed as in Example 1. Lyophilised hydrolysate was used for tests following to protocol described in example 3.

### 1.5. Porcine Spinal Cord Pancreatine Hydrolysate

Fresh porcine spinal cord was processed and hydrolysed following the method described in Example 1, except that pancreatine (Sigma-Aldrich) was used for hydrolysis instead of pepsin while the pH of the reaction was maintained at 8.2. After hydrolysis which was carried out using the time and temperature parameters the same as in Example 1, the reaction solution was acidified to pH 2.5 by use of diluted hydrochloric acid and heated to 80° C. to deactivate the enzyme. The product of hydrolysis was further processed as in Example 1. Lyophilised hydrolysate was used for tests following to protocol described in example 3.

### 1.6. Whey Hydrolysate

Dried whey was processed and hydrolysed following the method as described in Example 1. Lyophilised hydrolysate was used for tests following the protocol described in example 3.

### 1.7. Bonito Hydrolysate

A pack of bonito muscle hydrolysate “ACE Peptide” from Nippon Supplement Inc. was diluted in 10 ml of water and centrifuged. Clear solution was frozen and lyophilised. Lyophilised hydrolysate was used for tests following to protocol described in example 3.

### 2. Test Conditions

The test conditions for all hydrolysates described in sections 1.1-1.7 of the present example were the same as described in Example 3. The control group consisted of chicks that received saline instead of study treatment.

### 3. Results

The results indicate that the administration of the Myelin Basic Protein Hydrolysate (MBP), Lamb Spinal Cord Protein Hydrolysate (LSCP), Veal Spinal Cord Protein Hydrolysate (VSCP) in the concentration range of 0.05 mg/ml to 0.2 mg/ml significantly increases remembering of training in day-old chicks. This was demonstrated by an increase in number of chicks avoiding pecking grains from 40%-45% in the control group (saline injection) to 70% at a concentration of 0.1 μg/μl of MBP, 79% for the concentration of 0.1 μg/μl of LSCP and over 86% at a concentration 0.1 μg/μl of VSCP. The results of “Strong Training” showed that said hydrolysates did not adversely affect the remembering of the training.

The results of experiments on administration of porcine spinal cord protein hydrolysates obtained by use of papain and pancreatine also showed increase in remembering of training in day-old chicks which was demonstrated by an increase in number of chicks avoiding pecking grains from about 40% in the control groups (saline injection control group, bonito proteins hydrolysate, whey proteins hydrolysate) to about 60% for the concentration of 0.2 μg/μl of hydrolysate obtained using pancreatine and about 55% at a concentration 0.2 μg/μl of the hydrolysate obtained using papain.

The obtained results demonstrate positive effect on memory caused by administration of all hydrolysates of proteins sourced from animal tissues of nervous system, irrespective of the protease used, although best results were obtained when pepsin was used. Also positive effect on memory was shown for Myelin Basic Protein, an isolated protein being one of protein components of the myelin substance. The most beneficial effect on memory and learning was shown for veal spinal cord proteins hydrolysate.

1. Enzymatic hydrolysate of proteins of animal tissue of nervous system for use in treatment of memory disorders.

2. Enzymatic hydrolysate for use according to claim 1, wherein said proteins are myelin proteins, preferably myelin basic protein.

3. Enzymatic hydrolysate for use according to claim 1, wherein said tissue is spinal cord tissue of an animal for slaughter, preferably porcine spinal cord tissue or lamb spinal cord tissue, more preferably veal spinal cord tissue.

4. Enzymatic hydrolysate for use according to claim 1, wherein said hydrolysate is obtained in the process of hydrolysis using papain or mammalian digestive enzyme, preferably pancreatine, more preferably pepsin.

5. Enzymatic hydrolysate for use according to claim 1, wherein said memory disorder is age associated memory loss or age associated impaired learning.

6. Composition for use in treatment of memory disorders characterised in that it comprises therapeutically effective amount of enzymatic hydrolysate according to claim 1, and one or more pharmaceutically acceptable carriers or excipients.

7. Use of enzymatic hydrolysate of proteins of animal tissue of nervous system as memory and learning enhancing agent in a food product.

8. Use according to claim 7, wherein said proteins are myelin proteins, preferably myelin basic protein.

9. Use according to claim 7, wherein said tissue is spinal cord tissue of an animal for slaughter, preferably porcine spinal cord tissue or lamb spinal cord tissue, more preferably veal spinal cord tissue.

10. Use according to claim 7, wherein said hydrolysate is obtained in the process of hydrolysis using papain or a mammalian digestive enzyme, preferably pancreatine, most preferably pepsin.

11. Use according to claim 7, wherein said food product is a dietary supplement.

12. Method of enhancing memory and learning in healthy animal wherein said method comprises administrating thereto an effective amount for enhancing memory and learning of enzymatic hydrolysate of proteins of animal tissue of nervous system.

13. Method according to claim 12, wherein said proteins are myelin proteins, preferably myelin basic protein.

14. Method according to claim 12, wherein said tissue is spinal cord tissue of an animal for slaughter, preferably porcine spinal cord tissue or lamb spinal cord tissue, more preferably veal spinal cord tissue.

15. Method according to claim 7, wherein said hydrolysate is obtained in the process of hydrolysis using papain or a mammalian digestive enzyme, preferably pancreatine, most preferably pepsin.
